(19) United States
(12) Patent Application Publication
(10) Pub. No.: US 2021/0093803 A1
(43) Pub. Date: Apr. 1, 2021

(54) ELECTRONIC INHALER WITH CONTACTLESS COMMUNICATION

(71) Applicant: Infineon Technologies AG, Neubiberg (DE)

(72) Inventors: Josef Haid, Graz (AT); Walter Kargl, Graz (AT); Armin Krieg, Graz (AT)

(21) Appl. No.: 16/587,574

(22) Filed: Sep. 30, 2019

(51) Int. Cl. A61M 11/04; G06K 7/08; G06K 7/10; G06F 21/31; A61B 5/1172; A61B 5/1171

(52) U.S. Cl. CPC A61M 11/042; G06K 7/087; G06K 7/10366; G06K 9/00013; A61B 5/1172; A61B 5/1176; G06F 21/31

(57) ABSTRACT

An electronic inhaler, including: an antenna; a contactless module configured to receive via the antenna from an authentication circuit of a removably-mounted liquid container, a liquid container authentication signal; and a processor configured to control an operational state of a heater, which is configured to vaporize a liquid of the removably-mounted liquid container, based on the liquid container authentication signal.

### BACKGROUND

An electronic inhaler is a hand-held device that uses a heater to vaporize a liquid to be inhaled by a user. The electronic inhaler may be a cigarette (e-cigarette), a medical device, or any other device that enables a user to inhale a vapor. The liquid may be provided within a liquid container that can be removably-mounted in or coupled with the electronic inhaler. The liquid may include propylene glycol, glycerin, and/or a similar organic liquid.

### BRIEF DESCRIPTION OF THE DRAWINGS

FIG. 1A illustrates a schematic diagram of an arrangement including an electronic inhaler and a removably-mounted liquid container in accordance with aspects of the disclosure.

FIG. 1B illustrates a schematic diagram of an arrangement including an electronic inhaler, which has a fingerprint sensor circuit, and a removably-mounted liquid container in accordance with aspects of the disclosure.

FIG. 1C illustrates a schematic diagram of an arrangement including an electronic inhaler, which has a facial characteristic sensor circuit, and a removably-mounted liquid container in accordance with aspects of the disclosure.

FIG. 1D illustrates a schematic diagram of an arrangement including an electronic inhaler and a removably-mounted liquid container, along with an external reader, in accordance with aspects of the disclosure.

FIG. 1E illustrates a schematic diagram of an arrangement including an electronic inhaler, which has a single-chip contactless module, and a removably-mounted liquid container, along with an external reader in accordance with aspects of the disclosure.

FIG. 1F illustrates a schematic diagram of an arrangement including an electronic inhaler, which has a dual-chip contactless module, and a removably-mounted liquid container, along with an external reader in accordance with aspects of the disclosure.

FIG. 1G is a flowchart of a method of operating an electronic inhaler in accordance with aspects of the disclosure.

FIG. 1H is a flowchart of a method of operating a liquid container in accordance with aspects of the disclosure.

FIG. 2A illustrates a schematic diagram of an arrangement including an electronic inhaler, along with an external reader, in accordance with aspects of the disclosure.

FIG. 2B illustrates a schematic diagram of an arrangement including an electronic inhaler, which has a fingerprint sensor circuit, along with an external reader in accordance with aspects of the disclosure.

FIG. 2C is a flowchart of a method of operating an electronic inhaler in accordance with aspects of the disclosure.

FIG. 3A illustrates a schematic diagram of an arrangement including an electronic inhaler, which has a fingerprint sensor circuit, in accordance with aspects of the disclosure.

FIG. 3B is a flowchart of a method of operating an electronic inhaler in accordance with aspects of the disclosure.

FIG. 4A illustrates a schematic diagram of an arrangement including an electronic inhaler, which has a facial characteristic sensor circuit, in accordance with aspects of the disclosure.

FIG. 4B is a flowchart of a method of operating an electronic inhaler in accordance with aspects of the disclosure.

### DETAILED DESCRIPTION

The present disclosure is directed to an electronic inhaler configured to communicate contactlessly with a removably-mounted liquid container and/or with an external reader. The electronic inhaler may also have a sensor circuit configured to enable authentication of an electronic inhaler user and/or a liquid container paired with the electronic inhaler.

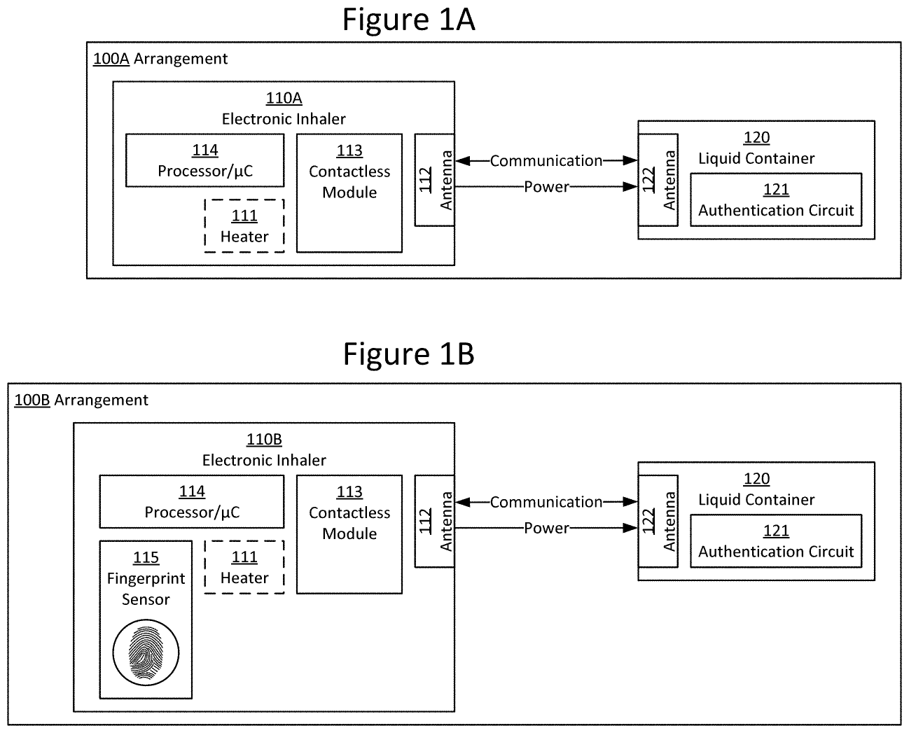

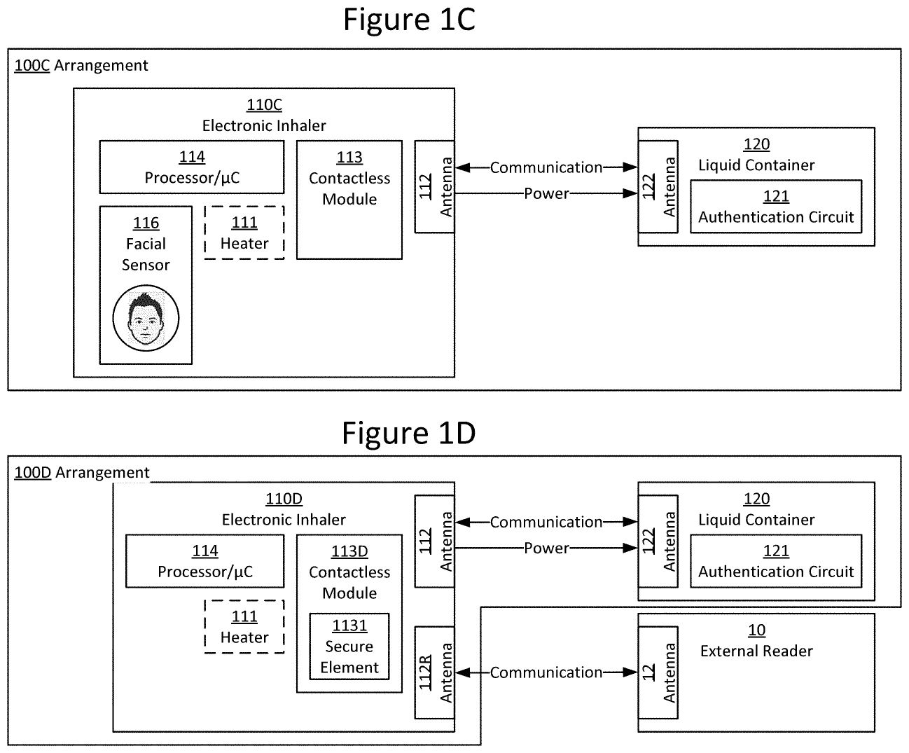

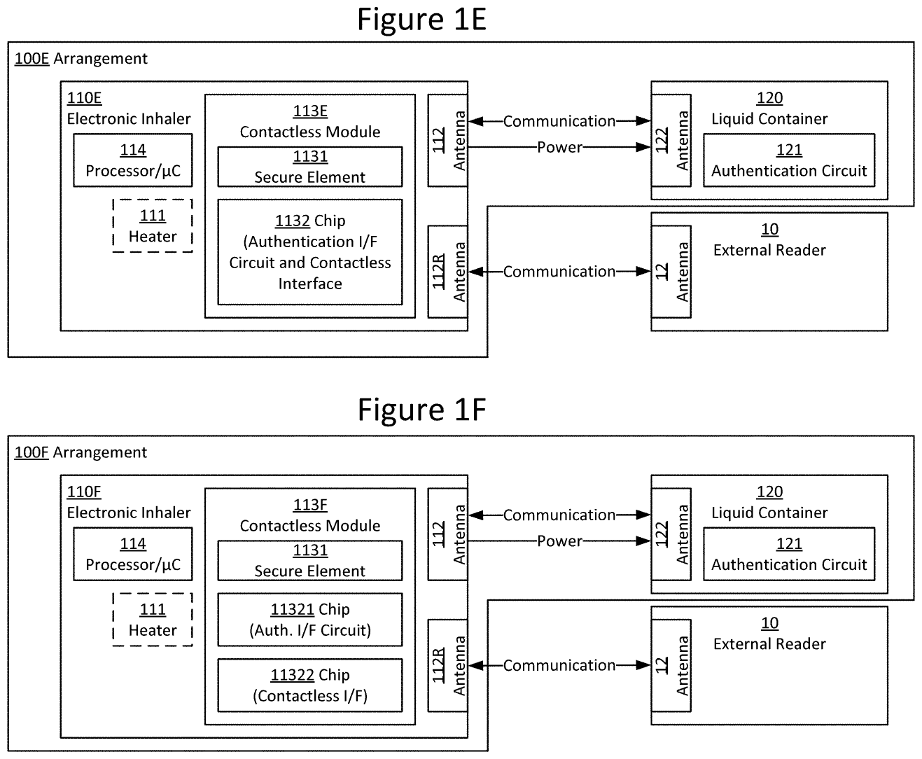

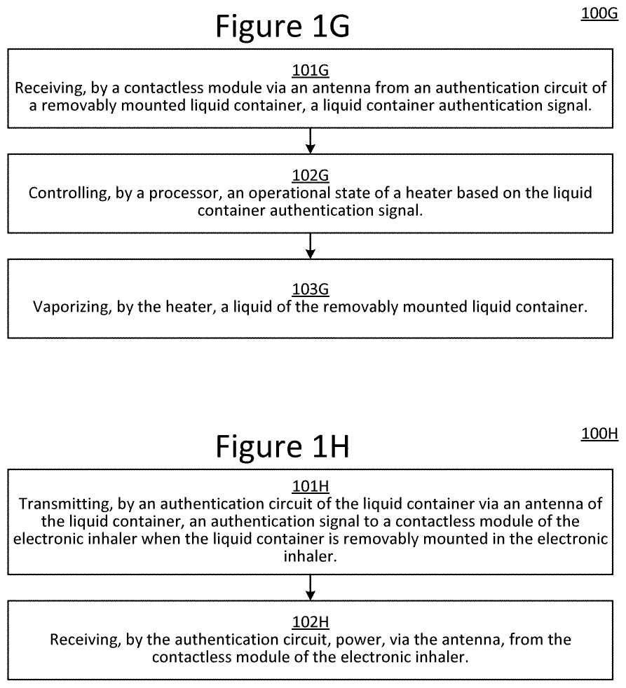

FIG. 1A illustrates a schematic diagram of an arrangement 100A including an electronic inhaler 110A and a removably-mounted liquid container 120 in accordance with aspects of the disclosure.

The electronic inhaler 110A comprises a heater 111, an antenna 112, a contactless module 113, and a processor 114. The removably-mounted liquid container 120 comprises an authentication circuit 121 and an antenna 122. The electronic inhaler 110A and the liquid container 120 communicate contactlessly with one another via coupling of their respective antennas 112, 122. The heater 111 is illustrated with a dashed box because at least a portion of the heater 111 may be comprised within the removably-mounted liquid container 120. For example, the removably-mounted liquid container 120 may have a heating coil which is activated by the electronic inhaler 110A inducing a current in the liquid container antenna 122.

The electronic inhaler 110A and the liquid container 120 may comprise other (or alternative) elements that have been omitted so as to not obscure other aspects of the disclosure. Also, the removably-mounted liquid container 120 is shown in the figures as separated from the electronic inhaler 110A, but in use, is physically connected in terms of the liquid, which flows from the removably-mounted liquid container 120 to the electronic inhaler 110A.

The authentication circuit 121 of the liquid container 120 is configured to transmit, via the antenna 122, a liquid container authentication signal to the electronic inhaler 110A. This transmission may occur optionally when the liquid container 120 is mounted in or coupled with the electronic inhaler 110A, or any other time as suitable. The contactless module 113 of the electronic inhaler 110A is configured to receive, via the antenna 112 coupling with the antenna 122, the liquid container authentication signal. The authentication signal may identify the liquid container 120 and/or be used to determine whether the liquid container 120 is authorized for use with the electronic inhaler 110A.

The processor 114 may perform the authentication to verify compatibility of the liquid container 120 with the electronic inhaler 110A, origin and distribution channels of the liquid container 120, and/or a quality of the liquid. Such authentication may be used to prevent product piracy, prevent use of expired liquid, etc. The processor 114 may be any kind of processor, controller, microcontroller, etc. as suitable, and may perform the authentication using a private key stored in the authentication circuit 121, for example. The authentication may be accomplished using asymmetric cryptographic algorithms such as Elliptic-Curve Cryptography (ECC), RSA, or the like, or alternatively, symmetric algorithms, such as Advanced Encryption Standard (AES), or the like.

The processor 114 is configured to control an operational state of the heater 111 to vaporize the liquid based on the liquid container authentication signal. The processor 114 is configured to verify, based on the liquid container authentication signal, whether the removably-mounted liquid container 120 is authorized for use with the electronic inhaler 110A. The removably-mounted liquid container 120 may therefore be authenticated by the processor 114 to ensure that the liquid container 120 is an authorized product rather than counterfeit. If authorization is successful, the processor may control the heater 111 to vaporize a liquid of the liquid container 120.

The contactless module 113 may be further configured to transmit, via coupling of the antennas 112, 122, power from the electronic inhaler 110A to the authentication circuit 121 of the removably-mounted liquid container 110A.

FIG. 1B illustrates a schematic diagram of an arrangement 100B including an electronic inhaler 110B, which has a fingerprint sensor circuit 115, and a removably-mounted liquid container 120 in accordance with aspects of the disclosure.

The arrangement 100B is similar to the arrangement 100A of FIG. 1A, except that the electronic inhaler 110B of the arrangement 100B additionally includes the fingerprint sensor circuit 115.

The fingerprint sensor circuit 115 uses a fingerprint to determine whether a fingerprint owner is authorized to use the electronic inhaler 110B and/or the liquid container 120. The fingerprint sensor circuit 115 is configured to sense a fingerprint by generating an image of or information about the ridges and valleys that make up the fingerprint, and then transmit a fingerprint image signal, which is based on the sensed fingerprint, to the processor 114. The processor 114 then processes the fingerprint image signal to identify and/or authenticate the fingerprint in order to grant or deny the fingerprint owner's use of the electronic inhaler 110B and/or the liquid container 120. The processor 114 may grant or deny use by controlling the operational state of the heater 111.

FIG. 1C illustrates a schematic diagram of an arrangement 100C including an electronic inhaler 110C, which has a facial characteristic sensor circuit 116, and a removably-mounted liquid container 120 in accordance with aspects of the disclosure.

The arrangement 100C is similar to the arrangement 100A of FIG. 1A, except that the electronic inhaler 110C of the arrangement 100C additionally includes the facial characteristic sensor circuit 116.

The facial recognition sensor circuit 116 uses a facial characteristic of a face to determine whether a person is authorized to use the electronic inhaler 110C and/or the liquid container 120. The facial recognition sensor circuit 116 is configured to sense a facial characteristic by generating an image of or information about the characteristic(s) that make(s) up the face, and then transmit a facial characteristic image signal, which is based on the sensed facial characteristic, to the processor 114. The processor 114 then processes the facial characteristic image signal to identify and/or authenticate the facial characteristic in order to grant or deny the facial characteristic owner's use of the electronic inhaler 110C and/or the liquid container 120. The processor 114 may grant or deny use by controlling the operational state of the heater 111. This facial characteristic may any facial characteristic, such as an ocular characteristic using, for example, iris and/or retinal scanning, though the disclosure is not limited in these respects. An iris scan is a biometric technique that generally uses visible and near-infrared light to take a high-contrast photograph of a person's iris. A retinal scan is a biometric technique that creates an image using unique patterns of person's retina blood vessels.

FIG. 1D illustrates a schematic diagram of an arrangement 100D including an electronic inhaler 110D and a removably-mounted liquid container 120, along with an external reader 10 in accordance with aspects of the disclosure.

The arrangement 100D is similar to the arrangement 100A of FIG. 1A, except that the electronic inhaler 110D is additionally configured to communicate with the external reader 10. The electronic inhaler 110D has a contactless module 113D with a secure element 1131, and a second antenna 112R for coupling with an antenna 12 of the external reader 10.

Although the electronic inhaler 110D is shown as having two antennas 112, 112R for respectively communicating with the liquid container 120 and the external reader 10, these two antennas 112, 112R may alternatively be replaced with a single antenna for communicating with both the liquid container 120 and the external reader 10. The contactless module 113D is configured to communicate with the external reader 10 via coupling of the antenna 112R and antenna 12. The secure element 1131, which is known, may provide a cryptographic service, an age-check of the user of the electronic-inhaler 110D and/or the liquid container 120, or a check of other security-relevant information.

The external reader 10 may be any of a smartcard reader, which may be a payment system reader, a transportation system reader, an access system reader, a power control reader, a data collection reader, an electronic inhaler software or firmware updater, an identification card reader, or the like. In the case of a payment system reader, the electronic inhaler 110D is used as a payment card together with the payment system reader. In the case of a transportation system reader, the electronic inhaler 110D is used as a transportation card together with the transportation system reader. External readers in general are known, and for the sake of brevity, their detailed descriptions are omitted here.

FIG. 1E illustrates a schematic diagram of an arrangement 100E including an electronic inhaler 110E and a removably-mounted liquid container 120, along with an external reader 10, in accordance with aspects of the disclosure.

The arrangement 100E is similar to the arrangement 100D of FIG. 1D, except that the arrangement 100E comprises an electronic inhaler 110E having a single-chip contactless module 113E. The single-chip 1132 of the contactless module 113E comprises both an authentication interface circuit enabling communication with the authentication circuit 121 of the removably-mounted liquid container 120, and a contactless interface enabling communication with the external reader 10.

FIG. 1F illustrates a schematic diagram of an arrangement 100F including an electronic inhaler 110F and a removably-mounted liquid container 120, along with an external reader 10, in accordance with aspects of the disclosure.

The arrangement 100F is similar to the arrangement 100D of FIG. 1D, except that the arrangement 100F comprises an electronic inhaler 110F having a dual-chip contactless module 113F. The dual-chip contactless module 113F comprises a first chip 11321 having an authentication interface circuit enabling communication with the authentication circuit 121 of the removably-mounted liquid container 120, and a second chip 11322 having an contactless interface enabling communication with the external reader 10. The first chip 11321 and the second chip 11322 may have dedicated antennas 112, 112R, or alternatively, a single antenna shared by both chips 11321, 11322.

FIG. 1G is a flowchart of a method 100G of operating an electronic inhaler 110 (110A, 1108, 100C, 110D, 110E, 110F) in accordance with aspects of the disclosure. The method 100G comprises the following steps.

In Step 101G, receiving, by a contactless module 113 via an antenna 112 from an authentication circuit 121 of a removably-mounted liquid container 112, a liquid container authentication signal. The liquid container authentication signal may be received when the liquid container 120 is mounted in the electronic inhaler 110 or another suitable time.

In Step 102G, controlling, by a processor 114, an operational state of a heater 111 based on the liquid container authentication signal. The processor 114 verifies, based on the liquid container authentication signal, whether the removably-mounted liquid container 120 is authorized for use with the electronic inhaler 110.

In Step 103G, vaporizing, by the heater 111, a liquid of the removably-mounted liquid container 120.

FIG. 1H is a flowchart of a method 100H of operating a liquid container 120 in accordance with aspects of the disclosure. The method 100H comprises the following steps.

In Step 101H, transmitting, by an authentication circuit 121 of the liquid container 120 via an antenna 121 of the liquid container 120, an authentication signal to a contactless module 113 of the electronic inhaler 110 when the liquid container 120 is removably-mounted in the electronic inhaler 110. The authentication signal indicates whether the liquid container 120 is authorized for use with the electronic inhaler 110.

In Step 102H, receiving, by the authentication circuit 121, power, via the antenna 121, from the contactless module 113 of the electronic inhaler 110.

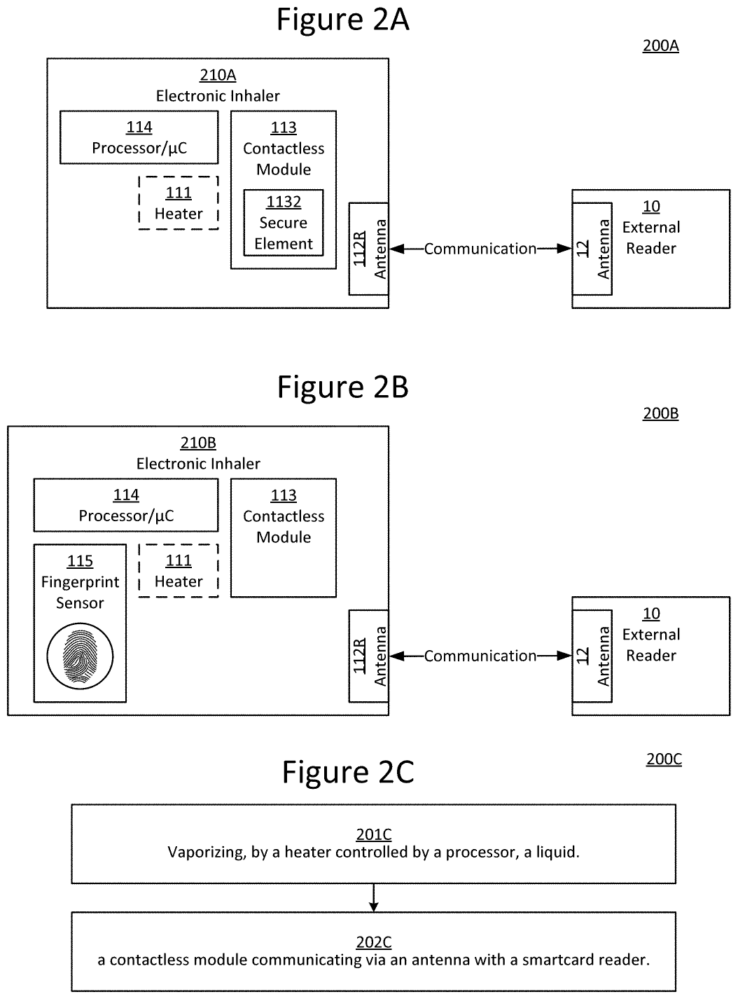

FIG. 2A illustrates a schematic diagram of an arrangement 200A including an electronic inhaler 210A, along with an external reader 10, in accordance with aspects of the disclosure.

The arrangement 200A is similar to the arrangement 100D of FIG. 1D, except that the removably-mounted liquid container 120, along with the corresponding electronic inhaler antenna 112, is not included. For the sake of brevity, the descriptions of the individual elements are not repeated here.

FIG. 2B illustrates a schematic diagram of an arrangement 200B of an electronic inhaler 210B, which has a fingerprint sensor circuit 115, along with an external reader 10, in accordance with aspects of the disclosure.

The arrangement 200B is similar to the arrangement 200A of FIG. 2A, except that the electronic inhaler 210B additionally includes a fingerprint sensor circuit 115, as described above with respect to FIG. 1B. For the sake of brevity, the descriptions of the individual elements are not repeated here.

FIG. 2C is a flowchart of a method 200C of operating the electronic inhaler 210 (210A and/or 210B) in accordance with aspects of the disclosure.

In Step 201C, vaporizing, by a heater 111 controlled by a processor 114, a liquid.

In Step 202C, a contactless module 113 of the electronic inhaler 210 communicating via an antenna 112 with a smartcard reader 10.

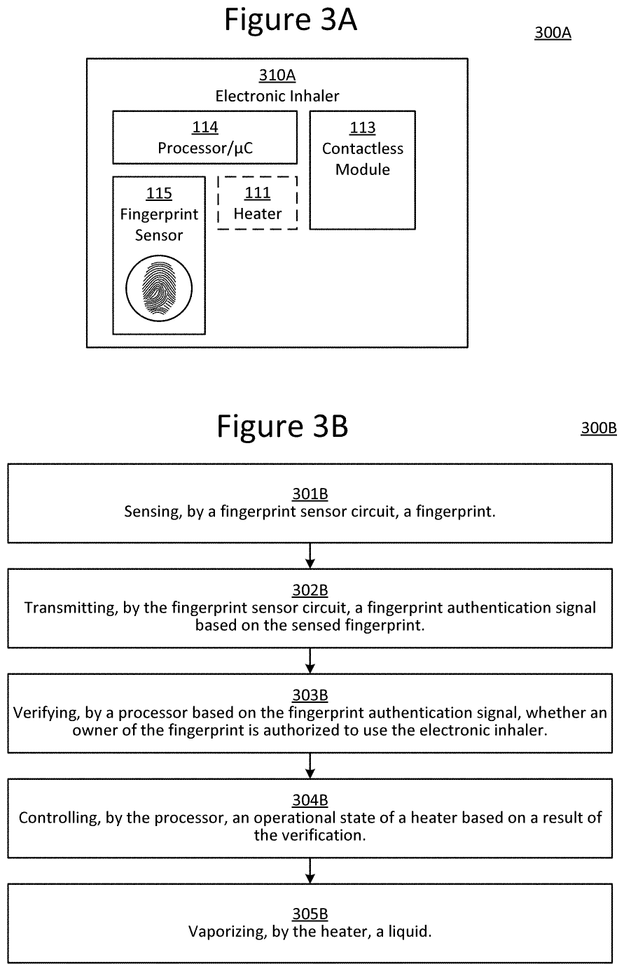

FIG. 3A illustrates a schematic diagram of an arrangement 300A including an electronic inhaler 310A, which has a fingerprint sensor circuit 115, in accordance with aspects of the disclosure.

The arrangement 300A is similar to the arrangement 100B of FIG. 1B, except that the removably-mounted liquid container 120, along with the corresponding electronic inhaler antenna 112, is not included. For the sake of brevity, the descriptions of the individual elements are not repeated here.

FIG. 3B is a flowchart of a method 300B of operating an electronic inhaler 310A in accordance with aspects of the disclosure.

At Step 301B, sensing, by a fingerprint sensor circuit 115, a fingerprint.

At Step 302B, transmitting, by the fingerprint sensor circuit 115, a fingerprint image signal based on the sensed fingerprint.

At Step 303B, verifying, by a processor 114 based on the fingerprint image signal, whether an owner of the fingerprint is authorized to use the electronic inhaler 310A.

At Step 304B, controlling, by the processor 114, an operational state of a heater 111 based on a result of the verification. The processor 114 may verify whether an owner of the fingerprint is authorized to use the electronic inhaler 310A and/or the electronic inhaler 310A with the particular liquid container 120 mounted therein.

At Step 305B, vaporizing, by the heater 111, a liquid.

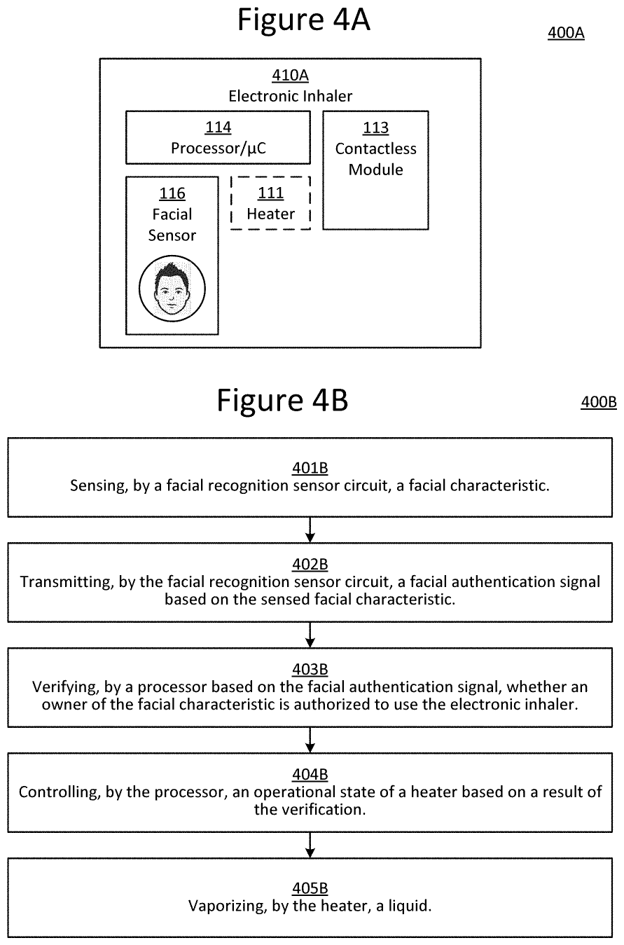

FIG. 4A illustrates a schematic diagram of an arrangement 400A including an electronic inhaler 410A, which has a facial characteristic sensor circuit 116, in accordance with aspects of the disclosure.

The arrangement 400A is similar to the arrangement 100B of FIG. 1B, except that the liquid container 120, along with the electronic inhaler antenna 112, is excluded. For the sake of brevity, the descriptions of the individual elements are not repeated here.

FIG. 4B is a flowchart of a method 400B of operating the electronic inhaler 410A in accordance with aspects of the disclosure.

At Step 401B, sensing, by a facial recognition sensor circuit 116, a facial characteristic. The facial characteristic may be an ocular characteristic.

At Step 402B, transmitting, by the facial recognition sensor circuit 116, a facial image signal based on the sensed facial characteristic.

At Step 403B, verifying, by a processor 114 based on the facial image signal, whether an owner of the facial characteristic is authorized to use the electronic inhaler 410A.

At Step 404B, controlling, by the processor 114, an operational state of a heater 111 based on a result of the verification. The processor 114 may verify whether an owner of the facial characteristic is authorized to use the electronic inhaler 410A and/or the electronic inhaler 410A with the particular liquid container 120 mounted therein.

At Step 405B, vaporizing, by the heater 11, a liquid.

The contactless communications described herein may be based on Near Field Communication (NFC), Bluetooth/Bluetooth-Low-Energy, WiFi, ZigBee, Z-Wave, and/or another related existing or upcoming protocol designed for users to control electronic devices.

The electronic inhaler described herein is advantageous in that it is a single device performing multiple functions. The results are reduced overall cost and increased convenience.

The techniques of this disclosure may also be described in the following examples.

### Example 1

An electronic inhaler, comprising: an antenna; a contactless module configured to receive via the antenna from an authentication circuit of a removably-mounted liquid container, a liquid container authentication signal; and a processor configured to control an operational state of a heater, which is configured to vaporize a liquid of the removably-mounted liquid container, based on the liquid container authentication signal.

### Example 2

The electronic inhaler of example 1, wherein the processor is further configured to verify, based on the liquid container authentication signal, whether the removably-mounted liquid container is authorized for use with the electronic inhaler.

### Example 3

The electronic inhaler of any combination of examples 1-2, wherein the contactless module is further configured to transmit, via the antenna, power to the authentication circuit of the removably-mounted liquid container.

### Example 4

The electronic inhaler of any combination of examples 1-3, wherein the contactless module is further configured to receive the liquid container authentication signal when the liquid container is mounted in the electronic inhaler.

### Example 5

The electronic inhaler of any combination of examples 1-4, further comprising: a fingerprint sensor circuit configured to sense a fingerprint and to transmit a fingerprint image signal based on the sensed fingerprint, wherein the processor is further configured to control the operational state of the heater based on the fingerprint image signal.

### Example 6

The electronic inhaler of any combination of examples 1-5, wherein the processor is further configured to verify, based on the fingerprint image signal, whether an owner of the fingerprint is authorized to use the electronic inhaler or the liquid container.

### Example 7

The electronic inhaler of any combination of examples 1-6, further comprising: a facial recognition sensor configured to sense a facial characteristic and to transmit a facial image signal based on the sensed facial characteristic, wherein the processor is further configured to control the operational state of the heater based on the facial image signal.

### Example 8

The electronic inhaler of any combination of examples 1-7, wherein the processor is further configured to verify, based on the facial image signal, whether an owner of the facial characteristic is authorized to use the electronic inhaler or the liquid container.

### Example 9

The electronic inhaler of any combination of examples 1-8, wherein the facial characteristic is an ocular characteristic.

### Example 10

The electronic inhaler of any combination of examples 1-9, wherein the contactless module is further configured to communicate with an external reader.

### Example 11

The electronic inhaler of any combination of examples 1-10, wherein the external reader is a smartcard reader.

### Example 12

The electronic inhaler of any combination of examples 1-11, wherein the external reader is a payment system reader, a transportation system reader, an access system reader, a power control reader, a data collection reader, an electronic inhaler software or firmware updater, or an identification card reader.

### Example 13

The electronic inhaler of any combination of examples 1-12, wherein the contactless module comprises a single-chip having both an authentication interface circuit to communicate with the authentication circuit of the removably-mounted liquid container, and a contactless interface to communicate with the external reader.

### Example 14

The electronic inhaler of any combination of examples 1-13, wherein the contactless module comprises a first chip having an authentication interface circuit to communicate with the authentication circuit of the removably-mounted liquid container, and a second chip having an contactless interface to communicate with the external reader.

### Example 15

An electronic inhaler, comprising: a processor configured to control a heater to vaporize a liquid of a removably-mounted liquid container; an antenna; and a contactless module configured to communicate via the antenna with a smartcard reader.

### Example 16

The electronic inhaler of example 15, wherein the smartcard reader is a payment system reader, a transportation system reader, an access system reader, a power control reader, a data collection reader, an electronic inhaler software or firmware updater, or an identification card reader.

### Example 17

The electronic inhaler of any combination of examples 15-16, further comprising: a fingerprint sensor circuit configured to sense a fingerprint and transmit a fingerprint image signal based on the sensed fingerprint; and a processor configured to control an operational state of the heater based on the fingerprint image signal.

### Example 18

The electronic inhaler of any combination of examples 15-17, wherein the processor is further configured to verify, based on the fingerprint image signal, whether an owner of the fingerprint is authorized to use the electronic inhaler or the liquid container.

### Example 19

An electronic inhaler, comprising: a fingerprint sensor circuit configured to sense a fingerprint and transmit a fingerprint image signal based on the sensed fingerprint; and a processor configured to verify, based on the fingerprint image signal, whether an owner of the fingerprint is authorized to use the electronic inhaler, and to control an operational state of a heater, which is configured to vaporize a liquid of a removably-mounted liquid container, based on a result of the verification.

### Example 20

The electronic inhaler of example 19, wherein the electronic inhaler has a liquid container removably-mounted therein, and the processor is configured to verify whether an owner of the fingerprint is authorized to use the electronic inhaler with the liquid container mounted therein.

### Example 21

An electronic inhaler, comprising: a facial recognition sensor configured to sense a facial characteristic and transmit a facial image signal based on the sensed facial characteristic; and a processor configured to verify, based on the facial image signal, whether an owner of the facial characteristic is authorized to use the electronic inhaler, and to control an operational state of a heater, which is configured to vaporize a liquid of a removably-mounted liquid container, based on a result of the verification.

### Example 22

The electronic inhaler of example 21, wherein the electronic inhaler has a liquid container removably-mounted therein, and the processor is configured to verify whether an owner of the facial characteristic is authorized to use the electronic inhaler with the liquid container mounted therein.

### Example 23

The electronic inhaler of any combination of examples 21-22, wherein the facial characteristic is an ocular characteristic.

### Example 24

A liquid container for an electronic inhaler, the liquid container comprising: an antenna; and an authentication circuit configured to transmit, via the antenna, an authentication signal to a contactless module of the electronic inhaler when the liquid container is removably-mounted in the electronic inhaler.

### Example 25

The liquid container of example 24, wherein the authentication circuit is configured to receive power, via the antenna, from the contactless module of the electronic inhaler.

### Example 26

The liquid container of any combination of examples 24-25, wherein the authentication signal indicates whether the liquid container is authorized for use with the electronic inhaler.

### Example 27

An electronic inhaler arrangement, comprising: a liquid container, comprising: a liquid container antenna; and an authentication circuit configured to transmit, via the liquid container antenna, a liquid container authentication signal; and an electronic inhaler, comprising: an electronic inhaler antenna; a sensor circuit configured to sense a fingerprint and transmit a fingerprint image signal based on the sensed fingerprint, and to sense a facial characteristic and transmit a facial image signal based on the sensed facial characteristic; and a contactless module configured to: receive via the electronic inhaler antenna, when the liquid container is removably-mounted in the electronic inhaler, the liquid container authentication signal; and communicate with an external reader; and a processor configured to: verify, based on the fingerprint image signal and the facial image signal, whether an owner of the fingerprint and the facial characteristic is authorized to use the electronic inhaler; and control an operational state of a heater, which is configured to vaporize a liquid of the removably-mounted liquid container, based on the liquid container authentication signal and a result of the verification.

### Example 28

A method of operating an electronic inhaler, comprising: receiving, by a contactless module via an antenna from an authentication circuit of a removably-mounted liquid container, a liquid container authentication signal; controlling, by a processor, an operational state of a heater based on the liquid container authentication signal; and vaporizing, by the heater, a liquid of the removably-mounted liquid container.

### Example 29

The example of example 28, further comprising: verifying, by the processor based on the liquid container authentication signal, whether the removably-mounted liquid container is authorized for use with the electronic inhaler.

### Example 30

The method of any combination of examples 28-29, further comprising: transmitting, by the contactless module via the antenna, power to the authentication circuit of the removably-mounted liquid container.

### Example 31

The method of any combination of examples 28-30, further comprising: receiving, by the contactless module, the liquid container authentication signal when the liquid container is mounted in the electronic inhaler.

### Example 32

The method of any combination of examples 28-31, further comprising: sensing, by a fingerprint sensor circuit, a fingerprint; transmitting, by the fingerprint sensor circuit, a fingerprint image signal based on the sensed fingerprint; and controlling, by the processor, the operational state of the heater based on the fingerprint image signal.

### Example 33

The method of any combination of examples 28-32, further comprising: verifying, by the processor based on the fingerprint image signal, whether an owner of the fingerprint is authorized to use the electronic inhaler or the liquid container.

### Example 34

The method of any combination of examples 28-33, further comprising: sensing, by a facial recognition sensor circuit, a facial characteristic; transmitting, by the facial recognition sensor circuit, a facial image signal based on the sensed facial characteristic; and controlling, by the processor, the operational state of the heater based on the facial image signal.

### Example 35

The method of any combination of examples 28-34, further comprising: verifying, by the processor based on the facial image signal, whether an owner of the facial characteristic is authorized to use the electronic inhaler or the liquid container.

### Example 36

The method of any combination of examples 28-34, wherein the facial characteristic is an ocular characteristic.

### Example 37

The method of any combination of examples 28-36, further comprising the contactless module communicating with an external reader.

### Example 38

The method of any combination of examples 28-37, wherein the external reader is a smartcard reader.

### Example 39

The method of any combination of examples 28-38, wherein the external reader is a payment system reader, a transportation system reader, an access system reader, a power control reader, a data collection reader, an electronic inhaler software or firmware updater, or an identification card reader.

### Example 40

The method of any combination of examples 28-39, wherein the contactless module comprises a single-chip having both an authentication interface circuit to communicate with the authentication circuit of the removably-mounted liquid container, and a contactless interface to communicate with the external reader.

### Example 41

The method of any combination of examples 28-40, wherein the contactless module comprises a first chip having an authentication interface circuit to communicate with the authentication circuit of the removably-mounted liquid container, and a second chip having an contactless interface to communicate with the external reader.

### Example 42

A method of operating an electronic inhaler, comprising: vaporizing, by a heater controlled by a processor, a liquid; and a contactless module communicating via an antenna with a smartcard reader.

### Example 43

The method of example 42, wherein the smartcard reader is a payment system reader, a transportation system reader, an access system reader, a power control reader, a data collection reader, an electronic inhaler software or firmware updater, or an identification card reader.

### Example 44

The method of any combination of examples 42-43, further comprising: sensing, by a fingerprint sensor circuit, a fingerprint; transmitting, by the fingerprint sensor circuit, a fingerprint image signal based on the sensed fingerprint; and controlling, by a processor, an operational state of the heater based on the fingerprint image signal.

### Example 45

The method of any combination of examples 42-44, further comprising: verifying, by the processor based on the fingerprint image signal, whether an owner of the fingerprint is authorized to use the electronic inhaler or the liquid container.

### Example 46

A method of operating an electronic inhaler, comprising: sensing, by a fingerprint sensor circuit, a fingerprint; transmitting, by the fingerprint sensor circuit, a fingerprint image signal based on the sensed fingerprint; verifying, by a processor based on the fingerprint image signal, whether an owner of the fingerprint is authorized to use the electronic inhaler; controlling, by the processor, an operational state of a heater based on a result of the verification; and vaporizing, by the heater, a liquid.

### Example 47

The method of example 46, wherein the electronic inhaler has a liquid container removably-mounted therein, and the method further comprises verifying, by the processor, whether an owner of the fingerprint is authorized to use the electronic inhaler with the liquid container mounted therein.

### Example 48

A method of operating an electronic inhaler, comprising: sensing, by a facial recognition sensor circuit, a facial characteristic; transmitting, by the facial recognition sensor circuit, a facial image signal based on the sensed facial characteristic; verifying, by a processor based on the facial image signal, whether an owner of the facial characteristic is authorized to use the electronic inhaler; controlling, by the processor, an operational state of a heater based on a result of the verification; and vaporizing, by the heater, a liquid.

### Example 49

The method of example 48, wherein the electronic inhaler has a liquid container removably-mounted therein, and the method further comprises verifying, by the processor, whether an owner of the facial characteristic is authorized to use the electronic inhaler with the liquid container mounted therein.

### Example 50

The method of any combination of examples 48-49, wherein the facial characteristic is an ocular characteristic.

### Example 51

A method of operating a liquid container of an electronic inhaler, comprising: transmitting, by an authentication circuit of the liquid container via an antenna of the liquid container, an authentication signal to a contactless module of the electronic inhaler when the liquid container is removably-mounted in the electronic inhaler.

### Example 52

The method of example 51, further comprising: receiving, by the authentication circuit, power, via the antenna, from the contactless module of the electronic inhaler.

### Example 53

The method of any combination of examples 51-52, wherein the authentication signal indicates whether the liquid container is authorized for use with the electronic inhaler.

### Example 54

A method of operating an electronic inhaler arrangement, comprising: transmitting, by an authentication circuit of a liquid container via a liquid container antenna, a liquid container authentication signal; receiving, by a contactless module of an electronic inhaler via an electronic inhaler antenna, when the liquid container is removably-mounted in the electronic inhaler, the liquid container authentication signal; sensing, by a sensor circuit of the electronic inhaler, a fingerprint; transmitting, by the sensor circuit, a fingerprint image signal based on the sensed fingerprint; sensing, by the sensor circuit, a facial characteristic;

transmitting, by the sensor circuit, a facial image signal based on the sensed facial characteristic; verifying, by a processor of the electronic inhaler, based on the fingerprint image signal and the facial image signal, whether an owner of the fingerprint and the facial characteristic is authorized to use the electronic inhaler; controlling, by the processor, an operational state of the heater based on the liquid container authentication signal and a result of the verification; vaporizing, by a heater of the electronic inhaler, a liquid of the liquid container; and communicating, by the contactless module, with an external reader.

While the foregoing has been described in conjunction with exemplary embodiment, it is understood that the term “exemplary” is merely meant as an example, rather than the best or optimal. Accordingly, the disclosure is intended to cover alternatives, modifications and equivalents, which may be included within the scope of the disclosure.

Although specific embodiments have been illustrated and described herein, it will be appreciated by those of ordinary skill in the art that a variety of alternate and/or equivalent implementations may be substituted for the specific embodiments shown and described without departing from the scope of the present disclosure. This disclosure is intended to cover any adaptations or variations of the specific embodiments discussed herein.

What is claimed is:

1. An electronic inhaler, comprising:
an antenna;
a contactless module configured to receive via the antenna from an authentication circuit of a removably-mounted liquid container, a liquid container authentication signal; and
a processor configured to control an operational state of a heater, which is configured to vaporize a liquid of the removably-mounted liquid container, based on the liquid container authentication signal.

2. The electronic inhaler of claim 1, wherein the processor is further configured to verify, based on the liquid container authentication signal, whether the removably-mounted liquid container is authorized for use with the electronic inhaler.

3. The electronic inhaler of claim 1, wherein the contactless module is further configured to transmit, via the antenna, power to the authentication circuit of the removably-mounted liquid container.

4. The electronic inhaler of claim 1, wherein the contactless module is further configured to receive the liquid container authentication signal when the liquid container is mounted in the electronic inhaler.

5. The electronic inhaler of claim 1, further comprising:
a fingerprint sensor circuit configured to sense a fingerprint and to transmit a fingerprint image signal based on the sensed fingerprint,
wherein the processor is further configured to control the operational state of the heater based on the fingerprint image signal.

6. The electronic inhaler of claim 5, wherein the processor is further configured to verify, based on the fingerprint image signal, whether an owner of the fingerprint is authorized to use the electronic inhaler or the liquid container.

7. The electronic inhaler of claim 1, further comprising:
a facial recognition sensor circuit configured to sense a facial characteristic and to transmit a facial image signal based on the sensed facial characteristic,
wherein the processor is further configured to control the operational state of the heater based on the facial image signal.

8. The electronic inhaler of claim 7, wherein the processor is further configured to verify, based on the facial image signal, whether an owner of the facial characteristic is authorized to use the electronic inhaler or the liquid container.

9. The electronic inhaler of claim 7, wherein the facial characteristic is an ocular characteristic.

10. The electronic inhaler of claim 1, wherein the contactless module is further configured to communicate with an external reader.

11. The electronic inhaler of claim 10, wherein the external reader is a smartcard reader.

12. The electronic inhaler of claim 10, wherein the external reader is a payment system reader, a transportation system reader, an access system reader, a power control reader, a data collection reader, an electronic inhaler software or firmware updater, or an identification card reader.

13. The electronic inhaler of claim 10, wherein the contactless module comprises a single-chip having both an authentication interface circuit to communicate with the authentication circuit of the removably-mounted liquid container, and a contactless interface to communicate with the external reader.

14. The electronic inhaler of claim 10, wherein the contactless module comprises a first chip having an authentication interface circuit to communicate with the authentication circuit of the removably-mounted liquid container, and a second chip having a contactless interface to communicate with the external reader.

15. An electronic inhaler, comprising:
a processor configured to control a heater to vaporize a liquid of a removably-mounted liquid container;
an antenna; and
a contactless module configured to communicate via the antenna with a smartcard reader.

16. The electronic inhaler of claim 15, wherein the smartcard reader is a payment system reader, a transportation system reader, an access system reader, a power control reader, a data collection reader, an electronic inhaler software or firmware updater, or an identification card reader.

17. The electronic inhaler of claim 15, further comprising:
a fingerprint sensor circuit configured to sense a fingerprint and transmit a fingerprint image signal based on the sensed fingerprint; and
a processor configured to control an operational state of the heater based on the fingerprint image signal.

18. The electronic inhaler of claim 17, wherein the processor is further configured to verify, based on the fingerprint image signal, whether an owner of the fingerprint is authorized to use the electronic inhaler or the liquid container.

19. An electronic inhaler, comprising:
a fingerprint sensor circuit configured to sense a fingerprint and transmit a fingerprint image signal based on the sensed fingerprint; and
a processor configured to verify, based on the fingerprint image signal, whether an owner of the fingerprint is authorized to use the electronic inhaler, and to control an operational state of a heater, which is configured to vaporize a liquid of a removably-mounted liquid container, based on a result of the verification.

20. The electronic inhaler of claim 19, wherein the electronic inhaler has a liquid container removably-mounted therein, and the processor is configured to verify whether an owner of the fingerprint is authorized to use the electronic inhaler with the liquid container mounted therein.

21. An electronic inhaler, comprising:
a facial recognition sensor circuit configured to sense a facial characteristic and transmit a facial image signal based on the sensed facial characteristic; and
a processor configured to verify, based on the facial image signal, whether an owner of the facial characteristic is authorized to use the electronic inhaler, and to control an operational state of a heater, which is configured to vaporize a liquid of a removably-mounted liquid container, based on a result of the verification.

22. The electronic inhaler of claim 21, wherein the electronic inhaler has a liquid container removably-mounted therein, and the processor is configured to verify whether an owner of the facial characteristic is authorized to use the electronic inhaler with the liquid container mounted therein.

23. The electronic inhaler of claim 21, wherein the facial characteristic is an ocular characteristic.

24. A liquid container for an electronic inhaler, the liquid container comprising:
an antenna; and
an authentication circuit configured to transmit, via the antenna, an authentication signal to a contactless module of the electronic inhaler when the liquid container is removably-mounted in the electronic inhaler.

25. The liquid container of claim 24, wherein the authentication circuit is configured to receive power, via the antenna, from the contactless module of the electronic inhaler.

26. The liquid container of claim 24, wherein the authentication signal indicates whether the liquid container is authorized for use with the electronic inhaler.

27. An electronic inhaler arrangement, comprising:
a liquid container, comprising:
a liquid container antenna; and
an authentication circuit configured to transmit, via the liquid container antenna, a liquid container authentication signal; and
an electronic inhaler, comprising:
an electronic inhaler antenna;
a sensor circuit configured to sense a fingerprint and transmit a fingerprint image signal based on the sensed fingerprint, and to sense a facial characteristic and transmit a facial image signal based on the sensed facial characteristic; and
a contactless module configured to:
receive via the electronic inhaler antenna, when the liquid container is removably-mounted in the electronic inhaler, the liquid container authentication signal; and
communicate with an external reader; and
a processor configured to:
verify, based on the fingerprint image signal and the facial image signal, whether an owner of the fingerprint and the facial characteristic is authorized to use the electronic inhaler; and
control an operational state of a heater, which is configured to vaporize a liquid of the liquid container, based on the liquid container authentication signal and a result of the verification.
